(19) United States
(12) Patent Application Publication
(10) Pub. No.: US 2026/0157972 A1
(43) Pub. Date: Jun. 11, 2026

(54) IMAGING-GUIDED BIORESORBABLE ACOUSTIC HYDROGEL MICROROBOTS

(71) Applicant: CALIFORNIA INSTITUTE OF TECHNOLOGY, Pasadena, CA (US)

(72) Inventors: Hong HAN, PASADENA, CA (US); Xiaotian MA, PASADENA, CA (US); Wei GAO, SAN MARINO, CA (US)

(21) Appl. No.: 19/181,177

(22) Filed: Apr. 16, 2025

Related U.S. Application Data
(60) Provisional application No. 63/635,882, filed on Apr. 18, 2024.

(51) Int. Cl. A61K 9/50; A61K 9/00; A61K 41/00; A61K 47/69; A61K 49/22

(52) U.S. Cl. CPC A61K 9/5094; A61K 9/0009; A61K 9/5031; A61K 9/5089; A61K 41/00; A61K 47/6941; A61K 49/225

(57) ABSTRACT

A bioresorbable acoustic microrobot (BAMs) designed for precision medicine applications. Fabricated through two-photon polymerization, BAMs incorporate 7-diethylamino-3-thenolycoumarin, poly(ethylene glycol) diacrylate, pentaerythritol tetraacrylate, and Fe₃O₄ nanoparticles for magnetic steering, alongside encapsulated therapeutic agents. These microrobots feature an asymmetric dual-opening bubble-trapping cavity, enhancing acoustic propulsion and ultrasound imaging capabilities. The unique dual-surface layer chemistry ensures efficient operation in biological fluids, while hydrolysis-driven biodegradability minimizes residual harm. BAMs demonstrate sustained drug release, precise navigation, and effective tumor targeting, offering significant advancements over conventional microrobots in therapeutic interventions.

### CROSS-REFERENCE TO RELATED APPLICATIONS

This application claims the benefit of U.S. Provisional Application No. 63/635,882 filed on Apr. 18, 2024, the contents of which are incorporated herein by reference in their entirety.

### STATEMENT REGARDING FEDERALLY SPONSORED R&D

This invention was made with government support under Grant No. CBET1931214 awarded by the National Science Foundation. The government has certain rights in the invention.

### BACKGROUND

Micro- and nanorobots have emerged as promising tools in biomedical engineering, with potential to revolutionize applications such as disease diagnosis, targeted drug delivery, detoxification, and minimally invasive surgery. These devices are designed to navigate the complex and often inaccessible areas within the human body, offering precision in medical interventions. The diverse propulsion mechanisms employed by these microrobots range from local chemical fuels to external fields like ultrasound or light, as well as harnessing the natural motility of microorganisms. Despite these innovations, their practical application in vivo faces significant challenges, particularly concerning stable propulsion through complex biological environments, real-time imaging, precise control, and effective therapeutic delivery.

### BRIEF SUMMARY

Various embodiments of the present disclosure may discuss an acoustic microrobot, including a polymeric matrix, a dual-surface layer encapsulating the polymeric matrix, where the dual-surface layer includes an inner hydrophobic surface and an outer hydrophilic surface, and a cavity positioned around the center of the polymeric matrix, where the cavity includes one or more openings extending through the polymeric matrix, where the one or more openings are positioned to increase acoustic propulsion and microstreaming effects.

Further embodiments of the acoustic microrobot may also include where the polymeric matrix is a hydrogel matrix, which further includes poly(ethylene glycol) diacrylate (PEGDA) and pentaerythritol tetra acrylate (PETA). The acoustic microrobot may also include where the polymeric matrix further includes magnetic nanoparticles. The acoustic microrobot may also include where the polymeric matrix further includes encapsulated therapeutic agents. The acoustic microrobot may also include where the polymeric matrix entraps a gaseous bubble in an internal cavity. The acoustic microrobot may also include where the polymeric matrix is contained within the acoustic microrobot by the inner hydrophobic surface. The acoustic microrobot may also include where the cavity is a dual-opening cavity that includes two openings positioned with relation to each other to increase acoustic and microstreaming effects. The acoustic microrobot may also include a propulsion mechanism, where the propulsion mechanism is an acoustic propulsion mechanism. The acoustic microrobot may also include where the acoustic microrobot is fabricated using two-photon polymerization. The acoustic microrobot may also include where the inner hydrophobic layer is modified with trichloro(1H,1H,2H,2H-perfluorooctyl) silane. The acoustic microrobot may also include where the asymmetric surface (or modified surface) further includes a hydrophobic self-assembled monolayer (SAM) on the inner surface and a hydrophilic outer surface.

Various embodiments of the present disclosure may include a method of using a bioresorbable acoustic microrobot (BAM) for targeted therapeutic delivery. The method may include navigating the BAM through biological fluids utilizing acoustic propulsion, where an entrapped gaseous bubble oscillates at its resonant frequency to generate microstreaming vortices, controlling the movement of the BAM via magnetic fields, leveraging magnetic nanoparticles embedded in a polymeric matrix for steering, releasing encapsulated therapeutic agents from the BAM at a target site, facilitated by passive diffusion of the polymeric matrix, and monitoring the position and movement of the BAM using ultrasound imaging, enabled by the entrapped gaseous bubble serving as a contrast agent.

Further embodiments of the method may also include diffusing the BAM through hydrolysis after releasing the encapsulated therapeutic agents. The method may also include calibrating an acoustic frequency to match the resonant frequency of the entrapped gaseous bubble. The method may also include where controlling the movement of the BAM further includes adjusting the magnetic field's strength and direction to steer the BAM through biological fluids. The method may also include where releasing encapsulated therapeutic agents further includes synchronizing the release of the encapsulated therapeutic agents with the BAM's arrival at the target site. The method may also include incorporating additional encapsulated therapeutic agents into the polymeric matrix for single or multiple drug delivery.

Various embodiments of the present disclosure may include a BAM for targeted therapeutic delivery, including a hydrogel matrix comprising poly(ethylene glycol) diacrylate (PEGDA) and pentaerythritol tetra acrylate (PETA), embedded magnetic nanoparticles distributed within the hydrogel matrix, and encapsulated therapeutic agents disposed within the hydrogel matrix, a dual-surface layer encapsulating the hydrogel matrix, comprising an inner hydrophobic surface chemically modified with trichloro(1H, 1H,2H,2H-perfluorooctyl) silane, and an outer hydrophilic surface constructed from O₂ plasma etching, a dual-opening cavity disposed internal to the dual-surface layer may include an entrapped gaseous bubble, an asymmetric configuration where a geometric center of the entrapped gaseous bubble deviates from a geometric center of the BAM, and two openings positioned at an angle, in relation to each other, configured to improve acoustic propulsion and microstreaming effects.

Further embodiments of the BAM may also include a propulsion mechanism, where the propulsion mechanism is an acoustic propulsion mechanism that propels the BAM by oscillating the entrapped gaseous bubble at a specific frequency. The BAM may also include where the embedded magnetic nanoparticles are Fe₃O₄ nanoparticles. The BAM may also include where the embedded magnetic nanoparticles are uniformly distributed within the hydrogel matrix. The BAM may also include where the outer hydrophilic layer includes a gradient of hydrophilicity to reduce aggregation. The BAM may also include where the PEGDA and PETA composition ratio is 9:1.

The BAM may also include where the magnetic nanoparticles are Fe₃O₄ nanoparticles. The BAM may also include where the internal cavity is asymmetrically structured such that a geometric center of the gaseous bubble deviates from a geometric center of the BAM. The BAM may also include where the two openings of the dual-opening cavity are positioned at approximately a 90-degree angle with relation to each other. The BAM may also include where the propulsion mechanism propels the BAM by oscillating an entrapped gaseous bubble. The BAM may also include where oscillating the entrapped gaseous bubble at the entrapped gaseous bubble's resonant frequency generates microstreaming vortices around the cavity and produces a propulsive force in an opposite direction of flow. Other technical features may be readily apparent to one skilled in the art from the following figures, descriptions, and claims.

### BRIEF DESCRIPTION OF THE SEVERAL VIEWS OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram showing a bioresorbable acoustic microrobot (BAM), in accordance with various embodiments of the disclosed technology.

FIG. 2A is a diagram showing a BAM with a single opening cavity, in accordance with various embodiments of the disclosed technology.

FIG. 2B is a diagram showing a BAM with a dual-opening cavity, in accordance with various embodiments of the disclosed technology.

FIG. 3 is a diagram showing the propulsion behavior of dual-opening cavity BAMs in comparison with single-opening cavity BAMs, in accordance with various embodiments of the disclosed technology.

FIG. 4A is a diagram showing a BAM with the geometric center of the entrapped bubble at the geometric center of the BAM, in accordance with various embodiments of the disclosed technology.

FIG. 4B is a diagram showing a BAM with the geometric center of the entrapped bubble offset from the geometric center of the BAM, in accordance with various embodiments of the disclosed technology.

FIG. 5 is a diagram showing a deep-tissue ultrasound imaging of a BAM with the entrapped bubbles serving as contrast agents, in accordance with various embodiments of the disclosed technology.

FIG. 6 is a diagram showing an in vitro tumor spheroid treatment process using BAMs, in accordance with various embodiments of the disclosed technology.

FIG. 7 is a diagram showing a hydrolysis-based biodegradation of BAMs in biofluids, in accordance with various embodiments of the disclosed technology.

FIG. 8 is a diagram showing the preparation and in vivo applications of BAMs, in accordance with various embodiments of the disclosed technology.

FIG. 9 is a diagram showing time-dependent drug release from BAMs, in accordance with various embodiments of the disclosed technology.

The figures are not intended to be exhaustive or to limit the invention to the precise form disclosed. It should be understood that the invention can be practiced with modification and alteration, and the disclosed technology be limited only by the claims and the equivalents thereof.

### DETAILED DESCRIPTION

For microrobots to function effectively within the human body's biological milieu, they may exhibit reliable and steady propulsion, enhanced imaging contrast for visualization under deep tissue, precise control for accurate targeting of diseased regions, substantial payload capacity for therapy, and high biocompatibility with biodegradable characteristics to eliminate or minimize the need for surgical removal. High-resolution additive manufacturing and flexible design are significant factors for the clinical adoption of these technologies. However, integrating these features within the confines of micro- or nanoscale platforms has previously presented challenges due to their limited size.

Acoustically actuated microrobots may be promising due to the distinct advantages of acoustic propulsion, including safety, non-invasive operation, deep-tissue penetration, robust propulsive forces, rapid response, and untethered control. These attributes may align well with clinical demands. Microrobots that exploit bubble oscillations induced by acoustic waves offer a promising approach for in vivo manipulation. However, the deployment of these microrobots has previously been hindered by transient stability and short lifespan of encapsulated bubbles within biological fluids, as well as difficulty achieving precisely controlled propulsion in complex in vivo environments.

The development of microbubble-based microrobots has been pursued to address these challenges. These devices leverage the oscillation of gas-liquid interfaces to generate propulsive forces, however, their practical application has been limited by bubble stability issues. The stability and functionality of microbubbles are relevant for both propulsion and imaging applications. Existing strategies have explored surface engineering and structural design modification to prolong bubble retention. Despite these efforts, the use of bioresorbable materials in microrobots introduces additional complexities due to the hydrophilic nature of hydrogel and/or polymeric surfaces, which can complicate long-term bubble retention.

Embodiments of the present disclosure discuss a bioresorbable acoustic microrobot (BAM), which may be a significant advancement in precision medicine, addressing numerous challenges faced by traditional micro- and nanorobots. These BAMs may be designed for minimally invasive medical interventions and targeted therapeutic delivery, offering enhanced navigation and propulsion capabilities within the human body. The BAMs may be fabricated using two-photon polymerization, a sophisticated technique that allows for the integration of magnetic nanoparticles and therapeutic agents into a polymeric matrix. This integration may enable more precise control and efficient drug delivery, making BAMs a significant improvement from earlier technologies.

Embodiments of the BAM may include a dual-surface layer. The dual-surface layer may be two different treatments applied to the exterior surface of the polymeric matrix. For purposes of discussion, embodiments of the dual-surface layer will be made with reference to layers, however, implementations of these embodiments may include an exterior surface or shell of the polymeric matrix with distinct surface treatments on opposing sides. The inner layer of the BAM may be hydrophobic, which may improve microbubble retention as needed for propulsion. In contrast, the outer layer may be hydrophilic, minimizing aggregation and assisting with the facilitation of timely degradation. This dual-surface layer may ensure that the BAMs do not persist too long in the body post-treatment, addressing concerns about long-term residual harm associated with non-bioresorbable materials.

Embodiments of the BAM may include a single or dual-opening cavity that may improve their speed and stability during movement. This feature may allow the BAMs to trap gaseous bubbles, maintaining more consistent and efficient acoustic propulsion across various biological environments. Embodiments with a dual-opening cavity may have each of the two openings strategically positioned, with relation to each other, to optimize propulsion by leveraging the microstreaming effects generated when bubbles oscillate in response to acoustic waves.

Embodiments of the BAM may be biodegradable. BAMs may be designed to by hydrolysis-driven, which may allow them to safely dissolve after their therapeutic task is completed. This characteristic may eliminate or reduce the risk of long-term residual harm, making BAMs more suitable for a wide range of in vivo applications. In some embodiments, the polymeric matrix may be a hydrogel matrix, composed of poly(ethylene glycol) diacrylate (PEGDA) and pentaerythritol tetra acrylate (PETA), providing the structural integrity for encapsulating therapeutic agents and magnetic nanoparticles. Further embodiments may not be biodegradable.

Embodiments of the BAM may further be navigable via magnetic field. Magnetic navigation may allow for wireless tracking and control of the BAMs through magnetic fields. The integration of magnetic nanoparticles, such as Fe₃O₄, within the polymeric matrix or hydrogel matrix may enable the BAMs to be more precisely steered to specific sites within the body. This capability may enhance the precision of navigation, making BAMs a more effective tool for targeted therapeutic delivery.

The propulsion mechanism of BAMs may be based on acoustic propulsion, where oscillating entrapped bubbles generate microstreaming vortices. These vortices may produce a propulsive force that drives the microrobots in the opposite direction of flow. Embodiments with a dual-opening cavity design may position the openings at approximately 90-degree angles to maximize these effects. Furthermore, the oscillation of bubbles at their resonant frequency may enhance propulsion efficiency and facilitate real-time or near-real-time ultrasound imaging, as the entrapped bubbles may act as contrast agents.

Therapeutically, BAMs may be designed for targeted drug delivery, capable of navigating through biological fluids and releasing encapsulated therapeutic agents more precisely at the target site. The controlled release may be facilitated by the passive diffusion of the polymeric matrix, and the process can be synchronized with the BAMs arrival at the target site. BAMs may also support drug delivery by incorporating additional therapeutic agents into the matrix, offering versatility in treatment protocols. For example, BAMs may encapsulate more than one drug to be delivered to a single or multiple target delivery sites in the human body.

In vitro and in vivo testing of BAMs has demonstrated their capabilities across various biological settings. The BAMs have shown efficient propulsion in different biofluids, precise navigation, and effective drug delivery. These results underscore the potential of BAM technology to improve practices in disease diagnosis, drug delivery, detoxification, and minimally invasive surgery.

Embodiments of the present disclosure may integrate several advanced features that collectively improve their utility in the clinical setting. The dual-surface layer design, single or dual-opening cavity, biodegradability, and magnetic navigation are all relevant components that contribute to improved performance. These discussed embodiments may allow BAMs to navigate complex biological environments effectively, providing more precise and controlled therapeutic delivery while improving biocompatibility and safety.

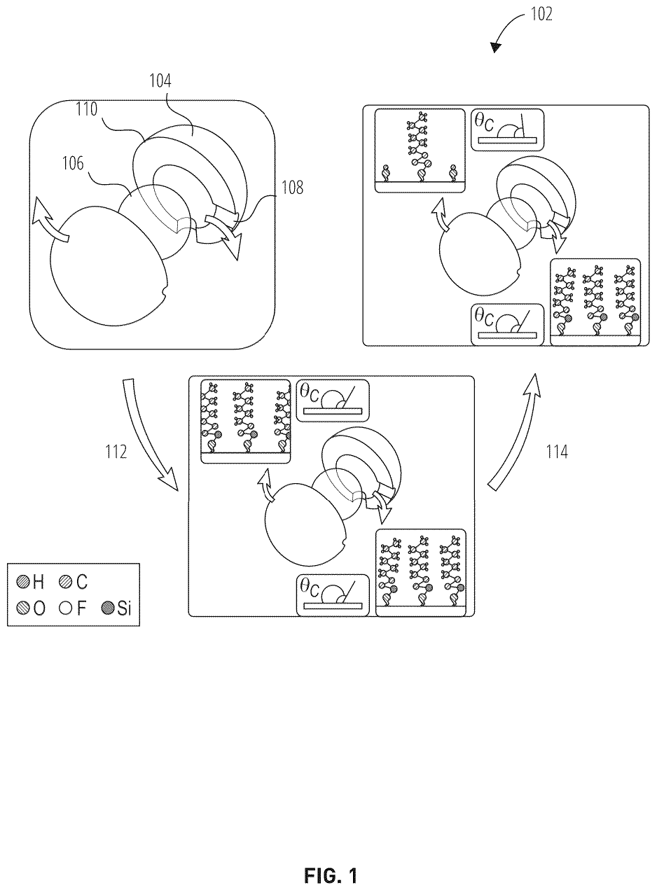

With respect to the figures, FIG. 1 is a diagram showing a bioresorbable acoustic microrobot 102 (BAM), in accordance with various embodiments of the disclosed technology. In response to the multifaceted challenges faced by conventional micro- and nanorobots, the present disclosure introduces a polymeric or hydrogel-based, imaging-guided, bioresorbable acoustic microrobot 102 designed for navigation with improved stability, precision, and control within the human body.

The BAM 102 may be modeled as a spherical shell of infinitesimal thickness immersed in a viscous liquid (the spherical shell may also be referred to as the spherical design herein). A gas bubble may be entrapped inside the shell, which may possess one or two circular openings on its surface. There may be a single or multiple gas bubbles entrapped within the polymeric matrix. The gas-liquid interface may be assumed to remain flat at equilibrium and may be pinned to the circular edge of the opening. The interface may vibrate upon actuation by an ultrasound field, generating acoustic streaming flow and thrust to propel the microrobot.

The polymeric matrix 104, which may be a hydrogel matrix, serves at the primary structure of the BAM 102. The polymeric matrix 104 may facilitate the integration of therapeutic agents and magnetic nanoparticles, such as Fe₃O₄, enabling more precise control of the BAM and its drug delivery. Embodiments with a hydrogel matrix may offer the additional benefit of biodegradability through hydrolysis, enhancing safety and biocompatibility by eliminating the need for surgical extraction post-treatment.

The integration of hydrogel or polymeric matrices as a primary component material of the BAMs presents substantial benefits for biomedical application in vivo. In a bladder cancer example, embedding the anticancer drug 5-FU into the hydrogel matrix of the BAMs improved the therapeutic efficiency compared with 5-FU alone. These BAMs may use a controlled-release mechanism that may prolong the bioavailability of the loaded drug, leading to sustained therapeutic activity and better outcomes.

The dual-opening cavity 108 may be designed to boost the propulsion efficiency of the BAM 102 in complex biofluids. This cavity design may facilitate the entrapment and oscillation of microbubbles, which may be relevant for generating propulsion and enhancing imagine contrast. The combination of asymmetric dual-opening cavity 108 and asymmetric surface hydrophobicity may be effective in stabilizing entrapped bubbles 106, improving robust operation in various biological fluids.

To improve in vivo operation, embodiments of the BAM 102 may utilize unique surface chemistry aimed at preserving the entrapped bubbles 106 for extended durations. For example, multiple day durations. To achieve this unique surface chemistry, a dual-step surface modification process may be used. Initially, BAMs 102 may be treated with fluorosilane to create a hydrophobic self-assembled monolayer (SAM) 112 on both the inner and outer surfaces of the shell. This hydrophobicity may be relevant for bubble entrapment but may predispose BAMs to self-aggregation. To mitigate this, a hydrophilic treatment may be applied via O₂ plasma etching 114 to the SAM-coated BAMs, which may achieve an outer hydrophilic surface while preserving the inner hydrophobic surface.

The dual-surface layer 110 modification may extend the entrapped bubbles' 106 life span within the BAMs, which may be relevant for propulsion and imaging. Unmodified hydrogel microrobots may lose their bubbles within one minute of immersion in biofluids due to the hydrophilic nature of the PEGDA film. For example, unmodified hydrogel microrobots may lose their bubbles within one minute of immersion in urine, attributed to the hydrophilic PEGDA film's 0° contact angle. In contrast, the modified BAMs, featuring an inner hydrophobic surface with a contact angle of approximately 133.4° and an outer hydrophilic surface with a contact angle of approximately 80.1°, can maintain microbubbles in human urine for up to 14 days. Other contact angles may exist, and the listed examples may be discussion purposes.

Furthermore, BAMs fabricated through high-resolution two-photon polymerization (TPP) may demonstrate improved bubble longevity. Traditional TPP resin IP-Dip-based microrobots typically lose their bubbles within an hour, while untreated hydrogel-based microrobots may lose their bubbles in less than one minute in biologically relevant fluids. In contrast, surface-treated BAMs may exhibit more robust bubble retention, with the majority of entrapped bubbles sustaining for multiple days. This extended bubble retention may not compromise the BAMs' mobility, which may remain more efficient (than conventional systems) even after prolonged immersion in various biofluids.

The incorporation of Fe₃O₄ nanoparticles (NPs) may enhance the superparamagnetic properties of BAMs, enabling more precise control over the acoustic propulsion of individual or swarms of BAMs through external magnetic fields. This capability, combined with the ability to navigate through untreated bodily fluids with varying viscosities, such as urine, gastrointestinal fluid, wound fluid, and whole fluid, underscores the versatility and potential for BAMs in biomedical applications. The trajectory of BAMs may also be influenced by the heterogeneity of biofluids, but the design may facilitate more effective navigation and therapeutic delivery of drugs to targeted sites.

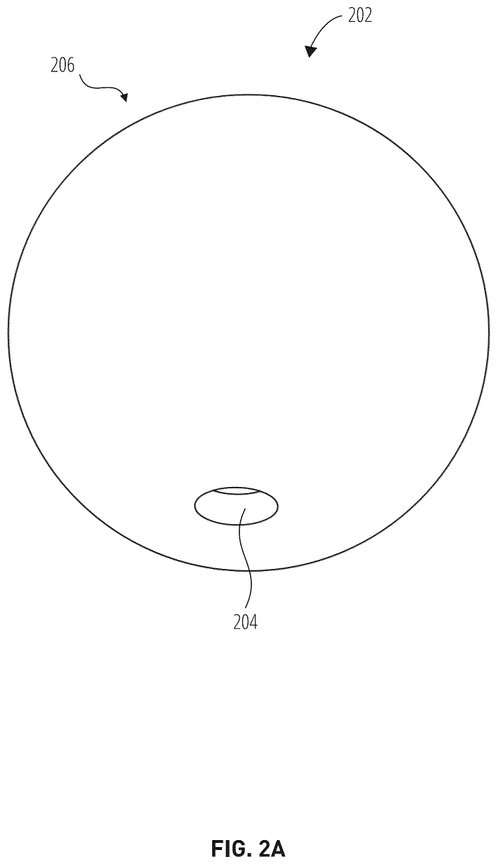

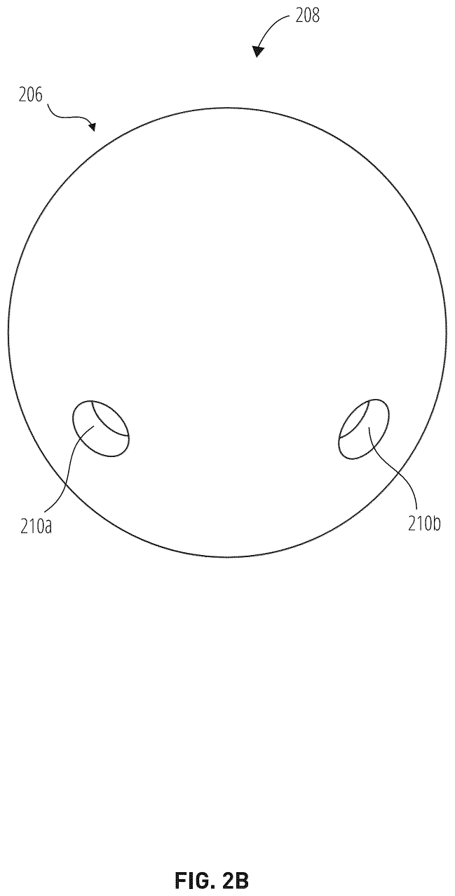

FIGS. 2A and 2B are diagrams showing a bioresorbable acoustic microrobot with a single-opening cavity 202 and a bioresorbable acoustic microrobot with a dual-opening cavity 208, in accordance with various embodiments of the disclosed technology. Embodiments of the BAMs, fabricated via TPP, may feature a spherical design 206 with an outer diameter ranging from 25 micrometers (μm) to 35 μm. For example, the diameter of the BAM may be approximately 30 μm. Both embodiments may include a single internal cavity, with the difference between the embodiments, as depicted between FIGS. 2A and 2B, being whether they include one or two openings. The internal cavity may be a spherical cavity designed to trap microbubbles in aqueous environments. The diameter of the internal cavity may range from 12 μm to 24 μm. For example, the diameter of the internal cavity may be 18 μm across.

In embodiments with a single-opening cavity 204, the BAM 202 may include a single internal cavity that opens to the surrounding environments. Under an ultrasound field, the entrapped bubbles within the BAM may pulsate, oscillating at their resonant frequency, which can be tuned by the size of the internal cavity. For example, for a spherical shape internal cavity, the resonant frequency may scale as:

$$f ∝ {( \frac{{n}^{3} γ}{{ρ r}^{3} {θ}_{0}^{3}} )}^{\frac{1}{2}}$$

In such scaling, r may be the radius of the entrapped bubble, y may be the adiabatic coefficient (which may range from 1.1 to 1.8, or in some embodiments be approximately 1.4), p may be the density of the fluid, θ₀=sin⁻¹(l/r) may be the azimuthal angle of the opening, l may be the width of the opening, and n may be the mode number. Embodiments with such scaling may utilize experimental or theoretical frequencies in accordance with below.

| Radius of | Width of | Experimental | Theoretical |
|---|---|---|---|
| bubble r | opening l | frequency | frequency |
| (μm) | (μm) | (kHz) | (kHz) |
| 7 | 6 | 500 | 491 |
| 8 | 6 | 430 | 472 |
| 9 | 6 | 300 | 428 |

Exemplary Resonant Frequencies for Printed BAMs with Single- or Dual-Opening Cavity

This oscillation of the gas-liquid interface may produce powerful microstreaming vortices around the opening, which may generate a propulsive force in the opposite direction of flow. However, the movement of bioresorbable acoustic microrobot with a single-opening cavity 202 may be influenced by Bjerknes forces, which may act perpendicularly to the boundary, often orienting the opening toward the boundary and resulting in unstable and/or less efficient translational movement.

In contrast, bioresorbable acoustic microrobot with a dual-opening cavity 208 may feature a first opening of the dual-opening cavity 210a and a second opening of the dual-opening cavity 210b. This configuration may allow BAMs to benefit from an additional propulsive force that runs parallel to the boundary, which may improve speed and stability of movement. The dual-opening design may allow for microstreaming patterns to be generated from both openings, enhancing their ability to generate propulsive thrust in additional directions compared to the single-opening design. Particle image velocimetry (PIV) analysis may be used to show pronounced microstreaming patterns from both openings in the dual-opening configuration, demonstrating the capability for more efficient propulsion.

The bioresorbable acoustic microrobot with a dual-opening cavity 208 can further be characterized by the angle between the openings, which may affect the propulsion efficiency. Experimental studies can show that the propulsion speed varies with the angle between the openings, reaching a maximum at approximately 90 degrees. This configuration may allow for optimal propulsive force generation, contributing to the improved maneuverability of BAMs. Additionally, triple-opening BAMs may exist, however, these embodiments may exhibit slower speeds that dual-opening configurations, due to partial cancellation of propulsion forces.

Both the single-opening and dual-opening BAMs may be designed to be bioresorbable, utilizing the polymeric or hydrogel matrix. The polymeric matrix may serve as the structural component, allowing for incorporation of therapeutic agents and magnetic nanoparticles such as Fe₃O₄, which may enhance the BAMs' functionality and control. The use of bioresorbable materials may improve the BAMs microrobots ability to safely degrade within the body after completing their therapeutic functions, minimizing the risk of long-term residual harm.

The BAMs' performance and bubble retention capabilities may be experimentally validated in various biological fluids. The dual-opening design, combined with asymmetric surface hydrophobicity (as discussed below), may be effective in stabilizing microbubbles and improving the robust operation within complex biological fluids. These features may enable BAMs to navigate through untreated body fluids with varying viscosities, demonstrating their potential for real-world biomedical applications. The incorporation of Fe₃O₄ nanoparticles may improve the BAMs superparamagnetic properties, allowing for more precise control over their acoustic propulsion through external magnetic fields, further enhancing their applicability in targeted therapeutic interventions.

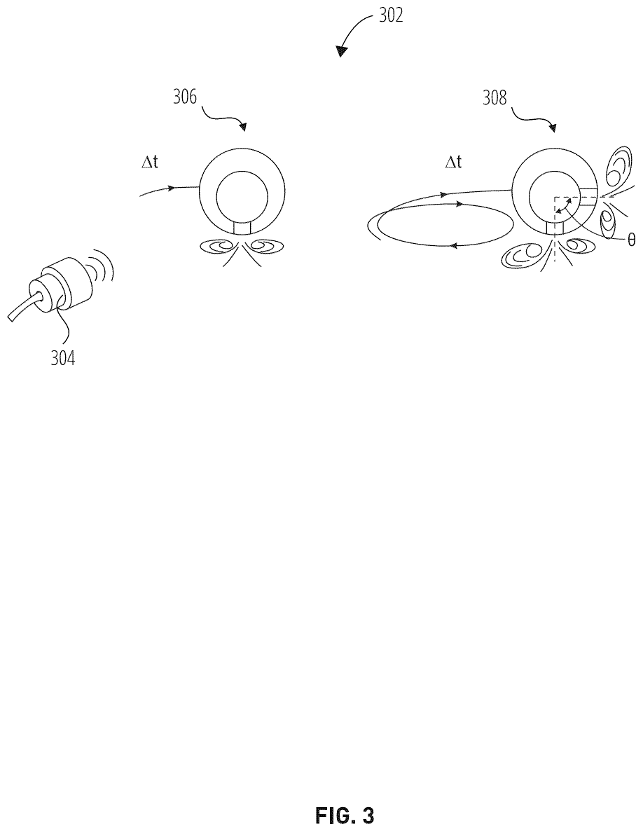

FIG. 3 is a diagram showing the propulsion behavior of dual-opening cavity BAMs in comparison with single-opening cavity BAMs 302, in accordance with various embodiments of the disclosed technology. This comparison may highlight the differences in propulsion efficiency and stability between the single-opening BAM 306 and the dual-opening BAM 308. The BAMs may be actuated by an ultrasound transducer 304, which can generate acoustic waves that interact with the entrapped bubbles within the BAMs to produce propulsion forces.

The single-opening BAM 306 may feature a single-opening to the internal cavity that opens to the surrounding environment. Under the influence of the ultrasound transducer 304, the entrapped bubbles may oscillate, generating microstreaming flows that produce propulsion forces. However, the movement of single-opening BAMs may be less stable and efficient due to the influence of secondary Bjerknes forces, which can act perpendicularly to boundaries and can lead to unstable translational movement. This limitation may be due to the unidirectional nature of the propulsion force generated from the single opening, which can be inconsistent in complex biological environments.

In contrast, the dual-opening BAM 308 may incorporate two openings to the internal cavity, allowing for the generation of additional propulsive forces that run parallel to boundaries, resulting in improved speed and stability of movement. The dual-opening design may facilitate more pronounced streaming flows from both openings, enhancing propulsion efficiency and enabling more effective navigation within biological environments. Experimental observations may show that dual-opening BAMs achieve higher propulsion speeds compared to single-opening BAMs, particularly when the openings are optimally angled to maximize propulsive force generation. This design advantage may underscore the dual-opening BAMs' potential for enhanced maneuverability and efficacy in biomedical applications, making them a more robust choice for tasks requiring precise control and navigation.

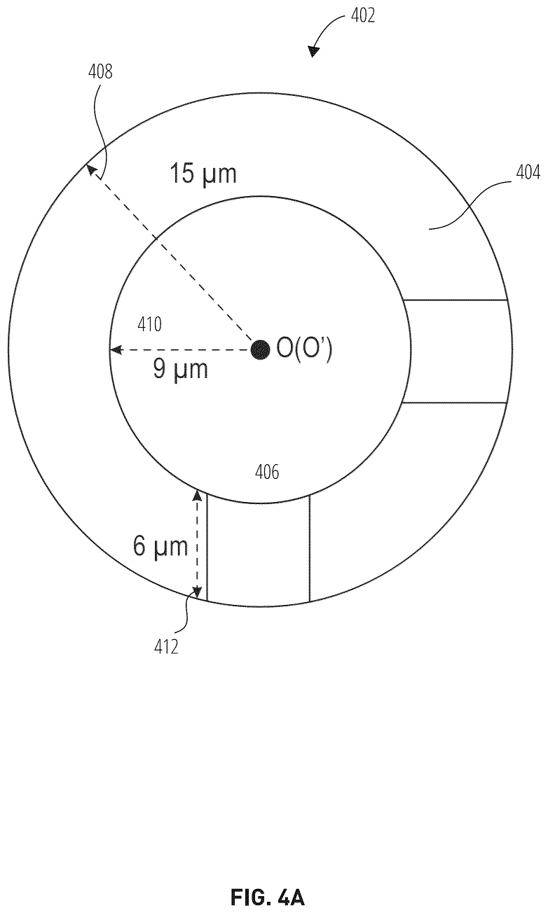

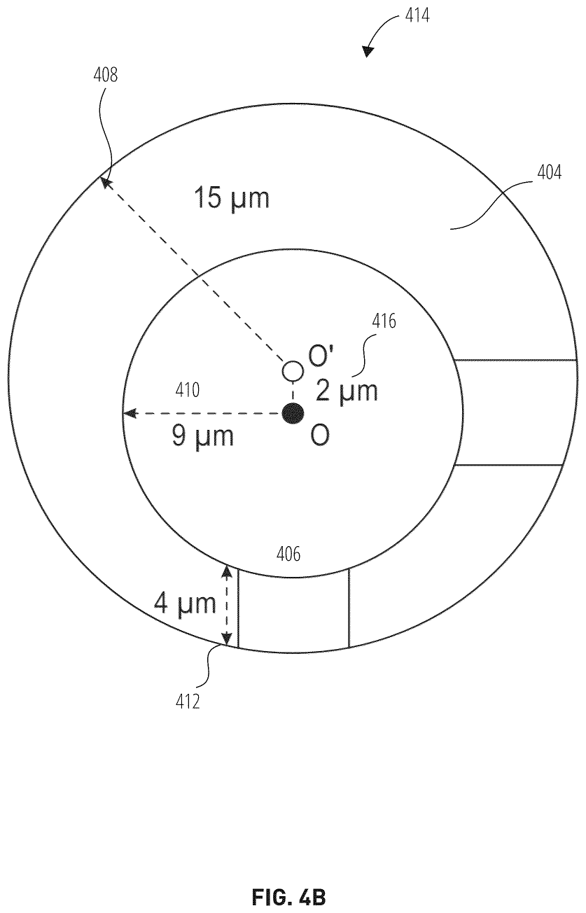

FIGS. 4A and 4B are diagrams showing a bioresorbable acoustic microrobot with an entrapped bubble in the geometric center 402 and a bioresorbable acoustic microrobot with an entrapped bubble offset from the geometric center 414, respectively, in accordance with various embodiments of the disclosed technology. The BAM 402 may be designed with the entrapped bubbles 406 positioned symmetrically within the polymeric matrix 404, which may have a radius of the radius of BAM 408 proportional to the disclosed diameters of the BAM, and an internal radius of entrapped bubble 410 proportional to the disclosed diameters of the internal cavity. This central alignment may provide uniform streaming flows around the BAM, facilitating consistent propulsion in various biological environments.

In contrast, bioresorbable acoustic microrobot with an entrapped bubble offset from the geometric center 414 may employ a geometric offset 416, such that the entrapped bubbles 406 are close to an edge of the BAM or reduces the depth of polymeric matrix 412 on a side of the BAM in comparison to the bioresorbable acoustic microrobot with an entrapped bubble in the geometric center 402. This geometric offset 416 position may allow the liquid-gas interfaces to be closer to the openings, resulting in more pronounced streaming flows. The asymmetry in the design may lead to improvement in the propulsion efficiency, enabling the BAM to achieve speeds exceeding those of the symmetric counterpart. This offset configuration may allow for increased interaction with the surrounding medium, which may enhance the streaming flow dynamics and subsequently the propulsion force.

The asymmetric design's enhanced propulsion performance may be particularly noticeable under certain experimental conditions. For example, tests may be conducted in phosphate-buffered saline (PBS) and indicate that the average speed of BAMs varied with the angle θ between their openings. A peak speed may be observed at approximately θ=90°, attributed to the optimal alignment of propulsive forces. The angle may allow the second opening's propulsive force to be maximized without a y-direction component, facilitating more efficient movement. Furthermore, when BAMs operate near a boundary, such as a wall, they may be influenced by the secondary Bjerknes forces, which can draw them closer to the boundary and guide their movement along it. This interaction may be relevant to the geometric offset in optimizing the microrobot's navigational capabilities in complex environments.

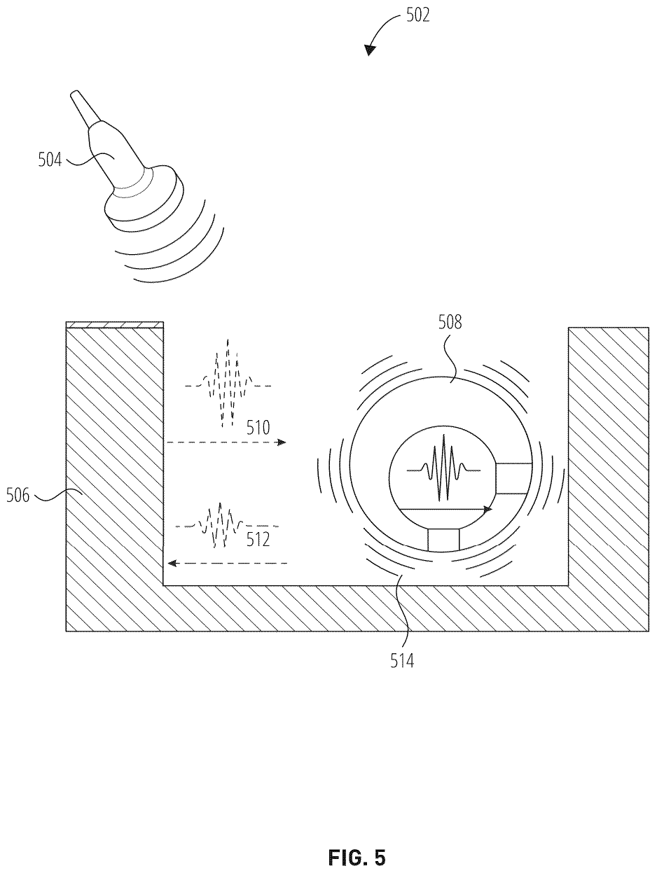

FIG. 5 is a diagram showing a deep tissue ultrasound imaging of a BAM 502 with the entrapped bubbles serving as contrast agents, in accordance with various embodiments of the disclosed technology. The deep tissue ultrasound imaging of a BAM 502 may utilize an ultrasound imaging probe 504 to visualize the BAM 508 within the biological environment 506. Biological environment 506 may encompass all relevant biological fluids discussed herein that exist in the human body, including but not limited to urine, gastrointestinal fluid, wound fluid, and whole blood. These fluids provide the medium through which the BAMs may navigate, and release encapsulated therapeutic agents to perform their therapeutic functions.

In the context of deep-tissue imaging, the entrapped bubbles within the BAM 508 may serve as contrast agents due to their unique acoustic properties. The phenomenon of emission 510 may occur when ultrasound waves interact with the entrapped bubbles, causing them to oscillate. This oscillation enhances the acoustic impedance mismatch between the entrapped bubbles and the surrounding biological environment 506, thereby increasing the ultrasound signal's strength and clarity. The resulting improved contrast may facilitate more precise localization and tracking of the BAM within the biological environment 506.

The processes of reflection 512 and transmission 514 may be relevant to the ultrasound imaging technique. Reflection 512 may refer to the bouncing back of ultrasound waves from the interface between the entrapped bubbles and the surrounding fluid, which provide information about the position and movement of the BAM 508. Transmission 514 may pertain to the passage of ultrasound waves through the BAM 508 and the surrounding biological environment 506, contributing to the overall imaging process by allowing deeper penetration and visualization within the biological environment 506. Together, these mechanisms may enable more effective real-time imaging and monitoring of BAMs, supporting their application in precision medicine for tasks such as targeted drug delivery and minimally invasive surgery.

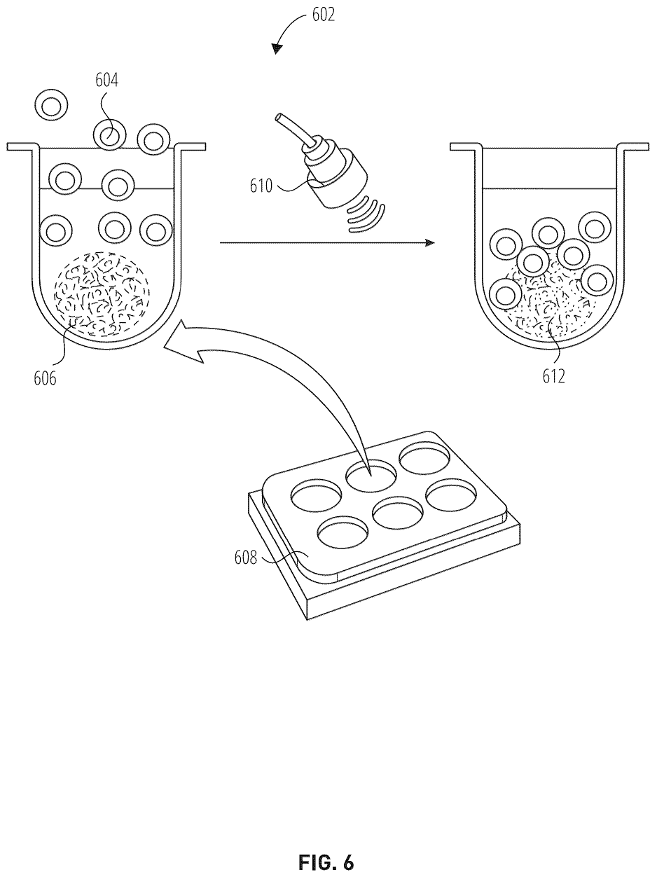

FIG. 6 is a diagram showing a method of an in vitro tumor spheroid treatment process using BAMs 602, in accordance with various embodiments of the disclosed technology. This method may leverage the unique properties of BAMs 604 to target and treat tumor spheroids 606. The BAMs may be designed to navigate the microplate culture 608 environment and utilize their acoustic propulsion capabilities to interact closely with tumor spheroids, which may enhance targeted drug delivery and treatment efficacy.

The BAMs 604 may be composed of a polymeric matrix, which may comprise poly(ethylene glycol) diacrylate (PEGDA), which is a US Food and Drug Administration-approved hydrogel material known for its nontoxicity and biodegradability. In aqueous environments, the hydrolysis of ester bonds within the cross-linked PEGDA framework may trigger the breakdown of polymer chains, leading to the gradual degradation of BAMs. This degradation process may be faster under alkaline conditions due to enhanced hydrolysis via saponification, ensuring that the BAMs can be safely degraded after delivering their therapeutic payload.

During the treatment process, the ultrasound propulsion mechanism 610 may be relevant to propulsion of the BAMs. It may actuate the BAMs, causing the entrapped bubbles to oscillate and propel the microrobots toward the tumor spheroids 606. This targeted movement may allow BAMs to deliver therapeutic agents directly to the tumor sites, maximizing the treatment's impact. The ultrasound propulsion mechanism 610 may facilitate more precise navigation and positioning of the BAMs within the microplate culture 608, ensuring more efficient interaction with the tumor spheroids.

To assess the effectiveness of the treatment, dead cell staining 612 may be employed. This process involves using specific dyes to identify and visualize dead cells within the tumor spheroids. The staining provides a relevant indication of the treatment's efficacy, as the presence of stained cells may correlate with successful targeting and destruction of tumor cells by the BAMs. The combination of biodegradable materials, targeted ultrasound propulsion, and effective cell staining makes this method a promising approach for in vitro cancer treatment and research.

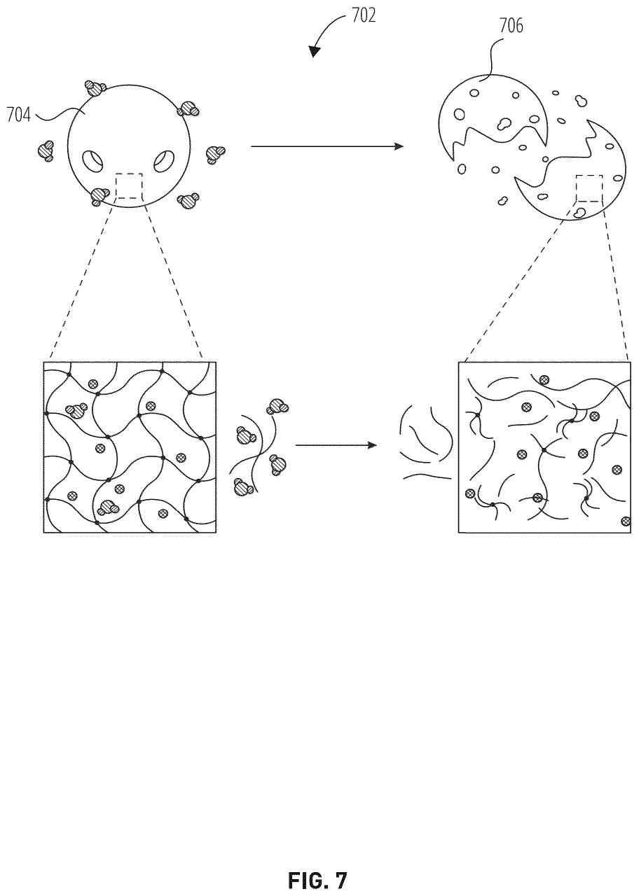

FIG. 7 is a diagram showing a hydrolysis-based biodegradation of BAMs in biofluids 702, in accordance with various embodiments of the disclosed technology. This process may highlight the capability of BAMs 704 to degrade safely within various biological environments after completing their therapeutic functions, ensuring biocompatibility, and minimizing the risk of long-term residual harm.

The biodegradation of BAMs 704 may be facilitated by the hydrolysis of ester bonds within the poly(ethylene glycol) diacrylate (PEGDA) matrix. This hydrogel material, approved by the US Food and Drug Administration, undergoes hydrolysis in aqueous environments, leading to the breakdown of polymer chains. The degradation process may be accelerated under alkaline conditions due to saponification, allowing for more rapid disintegration of the BAMs when necessary. This ensures that after performing their intended function, BAMs can be safely absorbed or eliminated by the body.

As BAMs 704 undergo hydrolysis within biological fluids 702, they transition into degraded BAMs 706. The ability to degrade more efficiently in fluids such as urine, gastrointestinal fluid, wound fluid, and whole blood, improves their versatility and safety in clinical applications. The hydrolysis process not only aids in eliminating BAMs post-treatment but also enhances their overall safety profile, making them a promising option for in vivo therapeutic applications.

The hydrolysis-based biodegradation mechanism is a relevant factor in the design and application of BAMs, as it ensures that the microrobots do not persist in the body, reducing the need for surgical extraction and minimizing potential complications associated with foreign material retention. This feature of BAMs significantly contributes to their appeal in precision medicine, offering a balance between therapeutic efficacy and biocompatibility.

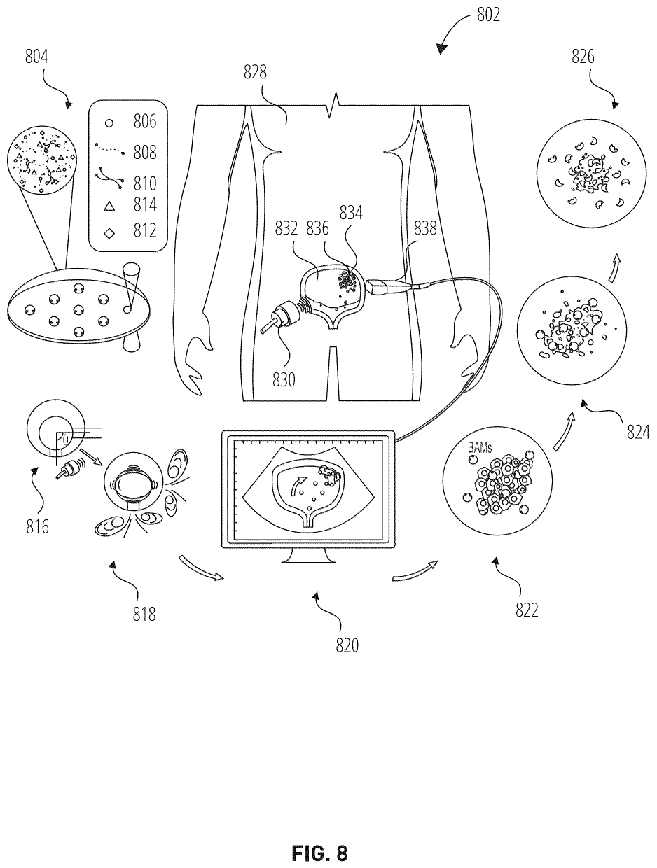

FIG. 8 is a diagram showing a method 802 of preparation and in vivo applications of BAMs, in accordance with various embodiments of the disclosed technology. The fabrication of BAMs may involve two-photon polymerization 804 (TPP), a sophisticated technique that enables the creation of highly detailed and functional microrobots. The composite material used in TPP may comprise 7-diethylamino-3-thenolycoumarin 806, poly(ethylene glycol) diacrylate 808 (PEGDA), and pentaerythritol tetraacrylate 810 (PETA). These materials may provide the structural integrity and biodegradability for the BAMs to function with improved efficacy within the human body 828.

Additional functional components may be integrated into the BAMs, including Fe₃O₄ 812 nanoparticles for magnetic steering. These nanoparticles may enhance the control and navigation of BAMs within biological environments, allowing for more precise targeting and manipulation. Therapeutic agents such as 5-fluorouracil 814 (5-FU) may also be incorporated into the BAMs, providing targeted drug delivery capabilities, particularly in the context of cancer treatment.

The BAMs design may incorporate an asymmetric single- or dual-opening bubble-trapping cavity 816, which may improve their acoustic propulsion 818 capabilities. The dual-opening configuration may allow for enhanced propulsion efficiency, as discussed, enabling BAMs to navigate complex biological fluids such as urine, gastrointestinal fluid, and whole blood over multiday durations. The asymmetric design may ensure that the liquid-gas interfaces are optimally positioned to generate powerful streaming flows, improving the propulsion speed and stability compared to single-opening configurations.

To achieve concurrent ultrasound imaging and acoustic propulsion of BAMs within soft tissue environments, a dual-probe approach may be used. This approach may comprise an ultrasound imaging probe 838 for real-time imaging of the bubbles and a focused ultrasound probe (FUS) designed for effective propulsion. The choice of FUS over piezoelectric disks may be informed by its strong field at a low input voltage, which may enhance its effectiveness and durability for the acoustic pressure amplitude attenuation through soft tissues. Adjusting the inner diameter of BAMs may allow for tuning of the resonant frequency of the entrapped bubbles to align with the FUS's center frequency, thereby maximizing operation efficiency.

To improve the functional lifespan of BAMs within biological environments, a unique dual-surface layer chemistry modification strategy may be employed. This may involve creating an inner hydrophobic surface to improve bubble retention within biological fluids and an outer hydrophilic surface to prevent microrobot aggregation and promote polymeric or hydrogel degradation. This dual-surface modification may ensure that BAMs maintain their functionality while minimizing potential complications associated with long-term retention in the body.

In addition to their propulsion capabilities, the microbubbles trapped by BAMs may serve as effective ultrasound imaging 820 contrast agents. The acoustic impedance mismatch between air and water, coupled with the volumetric oscillations induced by microbubbles when exposed to ultrasound waves, may enhance the ultrasound imaging contrast. This may allow for the movement and localization of BAMs 836 to be monitored in real-time with high spatiotemporal resolution using an ultrasound imaging probe 838 in combination with an ultrasound transducer 830.

The combination of advanced and stable ultrasound-driven acoustic propulsion 818 with concurrent ultrasound imaging 820 capabilities may emphasize the potential for in vivo biomedical applications. BAMs can be directed with precision via wireless magnetic navigation, making them suitable for tasks such as tumor targeting 822 and drug release 824. The hydrolysis-mediated biodegradation 826 of BAMs may improve their safe biological compatibility, reducing harmful residue risks after operation.

Preparation for the printing resin may involve adding PEG-functionalized Fe₃O₄ 812 nanoparticles to a PEGDA-PETA mixture to achieve the desired concentration. DETC, a photoinitiator, may be added to the resin and vortex mixed. 5-FU may be dissolved in dimethyl sulfoxide and added to the resin to achieve a final concentration, ensuring effective drug incorporation within BAMs.

For 3D printing of BAMs, a glass substrate may be immersed in 3-(trimethoxysilyl) propyl methacrylate solution, rinsed in water, and dried with nitrogen to enhance the surface adhesion of the prepared photoresist. The prepared resin (PEGDA-PETA/DETC/5-FU/Fe₃O₄) may be dropped onto the treated glass substrate and loaded into a 3D laser lithography tool. BAMs may be fabricated using oil immersion, and uncured photoresist may be removed using isopropyl alcohol before drying with nitrogen.

Surface modification of BAMs may involve activation in an oxygen plasma chamber and fluorosilanization with trichloro(1H,1H,2H,2H-perfluorooctyl) silane in a vacuum oven, followed by an oxygen plasma process. This treatment may enhance bubble retention and operational longevity within biological fluids, ensuring BAMs maintain their efficacy throughout their use in therapeutic applications.

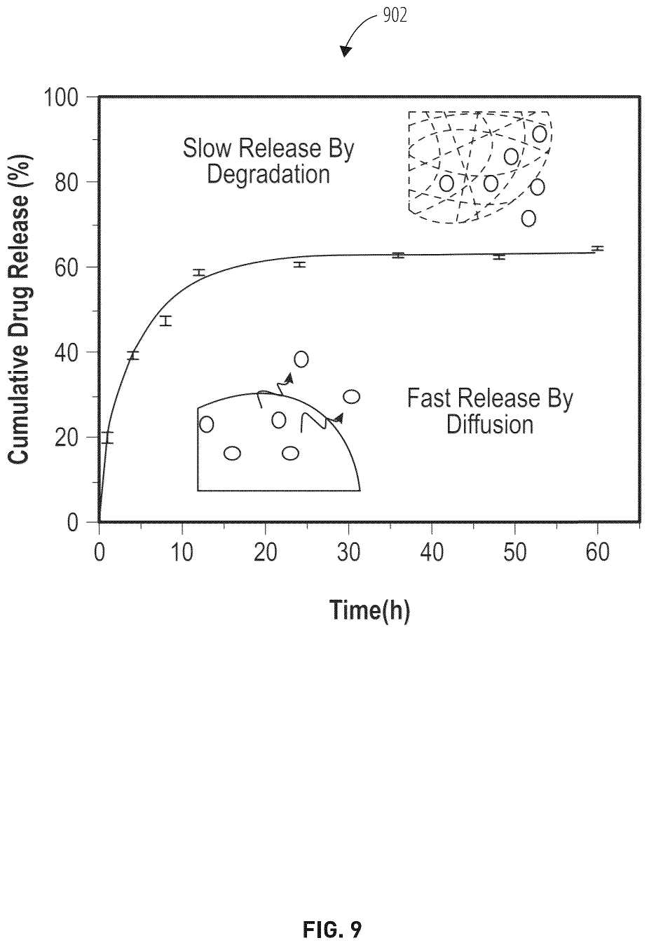

FIG. 9 is a diagram showing time-dependent cumulative drug release 902 from BAMs, in accordance with various embodiments of the disclosed technology. This graph may illustrate the release profile of therapeutic agents encapsulated within BAMs over a specified period, highlighting the effectiveness of BAMs in sustaining drug delivery.

The time-dependent cumulative drug release 902 may be characterized by the gradual diffusion and degradation of the polymeric matrix, which allows therapeutic agents like 5-fluorouracil (5-FU) to be released in a controlled manner. This sustained release mechanism may be relevant for maintaining therapeutic efficacy over extended durations, ensuring that the drug remains bioavailable in the target site. The release profile typically shows an initial burst phase followed by a prolonged release, which may be facilitated by the hydrolysis of the BAM's hydrogel matrix.

This method of drug delivery may be particularly advantageous in clinical settings, as it minimizes the frequency of administration and enhances patient compliance. The controlled release ensures that therapeutic levels of the drug are maintained, providing continuous treatment, and reducing the likelihood of side effects associated with peak doses. The graph serves as an example for understanding the pharmacokinetics of BAMs and optimizing their design for specific therapeutic applications.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

The terms “approximately” and “substantially” are used to account for variations that may occur due to manufacturing tolerances, measurement inaccuracies, and other practical considerations in implementing the described technology. The term “approximately” refers to a value or range that is close to the stated value but allows for minor deviations, typically within ±10%, unless otherwise specified, that do not materially affect the function or purpose of the invention. Similarly, the term “substantially” is used to indicate that a particular feature, characteristic, or result is largely present or achieved, with allowable variations without deviating from the intended scope and function of the invention. These terms should be interpreted in a manner consistent with the understanding of a person skilled in the art.

It should be noted that the terms “optimize,” “optimal” and the like as used herein can be used to mean making or achieving performance as effective or perfect as possible. However, as one of ordinary skill in the art reading this document will recognize, perfection cannot always be achieved. Accordingly, these terms can also encompass making or achieving performance as good or effective as possible or practical under the given circumstances, or making or achieving performance better than that which can be achieved with other settings or parameters.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An acoustic microrobot, comprising:
a polymeric matrix;
a dual-surface layer encapsulating the polymeric matrix, wherein the dual-surface layer comprises an inner hydrophobic surface and an outer hydrophilic surface; and
a cavity positioned internal to the polymeric matrix, wherein the cavity comprises one or more openings extending through the polymeric matrix and dual-surface layer, wherein the one or more openings are positioned to increase acoustic propulsion and microstreaming effects.

2. The BAM of claim 1, wherein the polymeric matrix is a hydrogel matrix, further comprising poly(ethylene glycol) diacrylate (PEGDA) and pentaerythritol tetra acrylate (PETA).

3. The BAM of claim 1, wherein the polymeric matrix further comprises magnetic nanoparticles.

4. The BAM of claim 3, wherein the magnetic nanoparticles are Fe₃O₄ nanoparticles.

5. The BAM of claim 1, wherein the polymeric matrix further comprises encapsulated therapeutic agents.

6. The BAM of claim 1, wherein the polymeric matrix entraps a gaseous bubble in an internal cavity.

7. The BAM of claim 6, wherein the internal cavity is asymmetrically structured such that a geometric center of the gaseous bubble deviates from a geometric center of the BAM.

8. The BAM of claim 1, wherein the polymeric matrix in contained within the BAM by the inner hydrophobic surface.

9. The BAM of claim 1, wherein the cavity is a dual-opening cavity that comprises two openings positioned with relation to each other to increase acoustic and microstreaming effects.

10. The BAM of claim 9, wherein the two openings of the dual-opening cavity are positioned at approximately a 90-degree angle with relation to each other.

11. The BAM of claim 1, further comprising a propulsion mechanism, wherein the propulsion mechanism is an acoustic propulsion mechanism.

12. The BAM of claim 11, wherein the propulsion mechanism propels the BAM by oscillating an entrapped gaseous bubble.

13. The BAM of claim 12, wherein oscillating the entrapped gaseous bubble at the entrapped gaseous bubble's resonant frequency generates microstreaming vortices around the cavity and produces a propulsive force in an opposite direction of flow.

14. The BAM of claim 1, wherein the BAM is fabricated using two-photon polymerization.

15. The BAM of claim 1, wherein the inner hydrophobic layer is modified with trichloro(1H, 1H,2H,2H-perfluorooctyl) silane.

16. The BAM of claim 1, wherein the dual-layer structure further comprises a self-assembled monolayer (SAM) on the inner hydrophobic layer.

17. A method of using a bioresorbable acoustic microrobot (BAM) for targeted therapeutic delivery, comprising:
navigating the BAM through biological fluids utilizing acoustic propulsion, wherein an entrapped gaseous bubble oscillates at its resonant frequency to generate microstreaming vortices;
controlling the movement of the BAM via magnetic fields, leveraging magnetic nanoparticles embedded in a polymeric matrix for steering;
releasing encapsulated therapeutic agents from the BAM at a target site, facilitated by passive diffusion of the polymeric matrix; and
monitoring the position and movement of the BAM using ultrasound imaging, enabled by the entrapped gaseous bubble serving as a contrast agent.

18. The method of claim 17, further comprising diffusing the BAM through hydrolysis after releasing the encapsulated therapeutic agents.

19. The method of claim 17, further comprising calibrating an acoustic frequency to match the resonant frequency of the entrapped gaseous bubble.

20. The method of claim 17, wherein controlling the movement of the BAM further comprises adjusting the magnetic field's strength and direction to steer the BAM through biological fluids.

21. The method of claim 17, wherein releasing encapsulated therapeutic agents further comprises synchronizing the release of the encapsulated therapeutic agents with the BAM's arrival at the target site.

22. The method of claim 17, further comprising incorporating additional encapsulated therapeutic agents into the polymeric matrix for multi-drug delivery.

23. A bioresorbable acoustic microrobot (BAM) for targeted therapeutic delivery, comprising:
a hydrogel matrix, comprising:
poly(ethylene glycol) diacrylate (PEGDA) and pentaerythritol tetra acrylate (PETA);
embedded magnetic nanoparticles distributed within the hydrogel matrix; and
encapsulated therapeutic agents disposed within the hydrogel matrix;
a dual-surface layer encapsulating the hydrogel matrix, comprising:
an inner hydrophobic surface chemically modified with trichloro(1H, 1H,2H,2H-perfluorooctyl) silane; and
an outer hydrophilic surface constructed from O₂ plasma etching;
a dual-opening cavity disposed internal to the dual-surface layer, comprising:
an entrapped gaseous bubble;
an asymmetric configuration wherein a geometric center of the entrapped gaseous bubble deviates from a geometric center of the BAM; and
two openings positioned at an angle, in relation to each other, configured to improve acoustic propulsion and microstreaming effects.

24. The BAM of claim 23, further comprising a propulsion mechanism, wherein the propulsion mechanism is an acoustic propulsion mechanism that propels the BAM by oscillating the entrapped gaseous bubble at a specific frequency.

25. The BAM of claim 23, wherein the embedded magnetic nanoparticles are Fe₃O₄ nanoparticles.

26. The BAM of claim 23, wherein the embedded magnetic nanoparticles are uniformly distributed within the hydrogel matrix.

27. The BAM of claim 23, wherein the outer hydrophilic layer comprises a gradient of hydrophilicity to reduce aggregation.

28. The BAM of claim 23, wherein the PEGDA and PETA composition ratio is 9:1.
